(19) United States
(12) Patent Application Publication
(10) Pub. No.: US 2025/0288869 A1
(43) Pub. Date: Sep. 18, 2025

(54) GAIT ANALYSIS METHOD AND DEVICE USING AUGMENTED REALITY INTERACTIVE SPORTS DEVICE

(71) Applicant: Twohands Interactive Inc., Busan (KR)

(72) Inventor: Wangyun YOO, Busan (KR)

(21) Appl. No.: 19/226,288

(22) Filed: Jun. 3, 2025

Related U.S. Application Data
(63) Continuation of application No. PCT/KR2023/003338, filed on Mar. 11, 2023.

(30) Foreign Application Priority Data
Jan. 12, 2023 (KR) 10-2023-0004697

(51) Int. Cl. A63B 24/00; A61B 5/11; A63B 71/06

(52) U.S. Cl. CPC A63B 24/0062; A61B 5/112; A61B 5/1126; A63B 2071/0638; A63B 2220/805

(57) ABSTRACT

Proposed are a gait analysis method and device using an augmented reality interactive sports device, the gait analysis method and device showing a gait path on a floor surface with the augmented reality interactive sports device and measuring foot position information of a user moving along the gait path to analyze and provide gait straightness and gait rhythm, thereby performing gait analysis conveniently with ease and at low cost by using the augmented reality interactive sports device used for various sports rather than an attached sensor or a controlled laboratory environment, and thus helping to identify poor gait habits and develop good gait habits.

### CROSS-REFERENCE TO RELATED APPLICATIONS

This application is a Continuation of Application No. PCT/KR2023/003338, filed on Mar. 11, 2023, which in turn claims the benefit of Korean Patent Application No. 10-2023-0004697, filed on Jan. 12, 2023. The entire disclosures of all these applications are hereby incorporated by reference.

### TECHNICAL FIELD

The present disclosure relates to a gait analysis method and device. More particularly, the present disclosure relates to a gait analysis method and device using an augmented reality interactive sports device.

### BACKGROUND ART

The description in this section merely provides background information on one embodiment of the present disclosure and does not constitute the related art.

Gait analysis is used for clinical decision-making in musculoskeletal and neurological disorders, post-treatment assessment of prostheses and assisting devices, and evaluation, many biomechanical studies, and measures multiple aspects of a gait to provide a comprehensive assessment, including kinematic analysis, dynamic electromyography, and energy expenditure measurement. In general, gait pattern extraction devices are commonly used for rehabilitation training to restore gait sensation of patients who suffer from gait disorders due to central nervous system damage such as stroke and cerebral palsy, or for gait posture correction training for users who need gait posture correction.

Conventional gait analysis methods require attached sensors (for example, inertial sensors such as gyro sensors and acceleration sensors) to be attached to a person's body to acquire information, and require the attached sensors to be attached for every measurement, which limits gait analysis in everyday life. In addition, gait analysis is expensive and difficult to perform because it requires the use of multiple sensors, a controlled laboratory environment, and equipment such as an analysis device.

In recent years, modern people have been spending more time sitting, which has led to an increase in scoliosis, pelvic distortion, and flat feet, and these problems affect gait. It is especially important to recognize and correct poor posture in children and teenagers early, helping them develop good gait habits. However, the conventional gait analysis methods are not readily accessible due to their cost, so the conventional gait analysis methods cannot be used as a casual check for good gait habits.

Therefore, if there is a technology that makes it easier and cheaper to do gait analysis, the technology will help to identify poor gait habits and develop good gait habits.

In the meantime, as a related art of the present disclosure, disclosed is Korean Patent No. 10-2233395 (title of invention: AUGMENTED REALITY INTERACTIVE SPORTS APPARATUS USING LIDAR SENSORS, registration date: 23 Mar. 2021).

The above-described background technology is technical information that has been possessed by the present inventor in order to derive the present disclosure or which has been acquired in the process of deriving the present disclosure, and can not necessarily be regarded as well-known technology which has been known to the public prior to the filing of the present disclosure.

### DISCLOSURE

### Technical Problem

The present disclosure has been made keeping in mind the above problems occurring in the previously proposed methods, and the present disclosure is directed to providing a gait analysis method and device using an augmented reality interactive sports device, the gait analysis method and device showing a gait path on a floor surface with the augmented reality interactive sports device and measuring foot position information of a user moving along the gait path to analyze and provide gait straightness and gait rhythm, thereby performing gait analysis conveniently with ease and at low cost by using the augmented reality interactive sports device used for various sports rather than an attached sensor or a controlled laboratory environment, and thus helping to identify poor gait habits and develop good gait habits.

However, the objectives of the present disclosure are not limited thereto, and there may be other objectives. Even if not explicitly mentioned, the objectives or effects that can be understood from the solutions or embodiments are also objectives of the present disclosure.

### Technical Solution

In order to achieve the above objectives, according to one aspect of the present disclosure, there is provided a gait analysis method using an augmented reality interactive sports device,

the gait analysis method

being embodied by the augmented reality interactive sports device configured to project a video onto a floor surface and track user motion by using a lidar sensor part to control interaction between content and a user, the gait analysis method including:
(1) showing a gait path on the floor surface through the augmented reality interactive sports device;
(2) measuring foot position information of the user moving along the gait path by using the lidar sensor part configured to laser scan a plane at a predetermined height from the floor surface; and
(3) analyzing gait straightness and gait rhythm from the foot position information of the user measured in the step (2) and measurement time.

Preferably, the gait path shown in the step (1) may be configured to pass, at least two times, a sensing area onto which the augmented reality interactive sports device projects the video, the sensing area being laser scanned by the lidar sensor part.

Preferably, the step (2) includes:
(2-1) a first measurement step of measuring the foot position information of the user moving along the gait path by using the lidar sensor part and recording the measured foot position information and the measurement time; and
(2-2) a second measurement step of measuring the foot position information of the user moving along the gait path by using the lidar sensor part and recording the measured foot position information and the measurement time after the first measurement step is terminated. In the step (2), gait measurement may be performed multiple times.

More preferably, in the step (2),

a state indication image indicating a progress state of the first measurement step and the second measurement step may be shown.

Preferably, the step (3) may include:
(3-1) analyzing a step length, a stride length, step time,

stride time, a gait velocity, and the number of steps per minute from the foot position information of the user measured in the step (2) and the measurement time;
(3-2) analyzing the gait straightness by using the foot position information of the user measured in the step (2) and graphing the gait straightness; and
(3-3) analyzing the gait rhythm on the basis of the step length and the step time analyzed in the step (3-1), and graphing the gait rhythm.

Preferably, the gait analysis method may further include: after the step (3),
(4) analyzing a gait stability index from the gait straightness and the gait rhythm analyzed in the step (3), and providing the gait stability index through the augmented reality interactive sports device.

In order to achieve the above objectives, according to one aspect of the present disclosure, there is provided a gait analysis device using an augmented reality interactive sports device,

the gait analysis device

transmitting and receiving data to and from the augmented reality interactive sports device configured to project a video onto a floor surface and track user motion by using a lidar sensor part to control interaction between content and a user, the gait analysis device including:

a gait path generation part configured to show a gait path on the floor surface through the augmented reality interactive sports device;

a gait measurement part configured to measure foot position information of the user moving along the gait path by using the lidar sensor part configured to laser scan a plane at a predetermined height from the floor surface; and

a gait analysis part configured to analyze gait straightness and gait rhythm from the foot position information of the user measured by the gait measurement part and measurement time.

Preferably,

the gait analysis device may further include an index

analysis part configured to analyze a gait stability index from the gait straightness and the gait rhythm, and provide the gait stability index through the augmented reality interactive sports device.

### Advantageous Effects

According to a gait analysis method and device using an augmented reality interactive sports device proposed in the present disclosure, a gait path is shown on a floor surface with the augmented reality interactive sports device and foot position information of a user moving along the gait path is measured to analyze and provide gait straightness and gait rhythm, thereby performing gait analysis conveniently with ease and at low cost by using the augmented reality interactive sports device used for various sports rather than an attached sensor or a controlled laboratory environment, and thus helping to identify poor gait habits and develop good gait habits.

Furthermore, various useful advantages and effects of the present disclosure are not limited to the above-described contents, and will be more easily understood in the process of describing specific embodiments of the present disclosure.

### DESCRIPTION OF DRAWINGS

FIG. 1 is a diagram illustrating the configuration of a gait analysis device using an augmented reality interactive sports device according to an embodiment of the present disclosure.

FIG. 2 is a diagram illustrating an augmented reality interactive sports device used for a gait analysis method and device using an augmented reality interactive sports device according to an embodiment of the present disclosure.

FIG. 3 is a diagram illustrating the flow of a gait analysis method using an augmented reality interactive sports device according to an embodiment of the present disclosure.

FIG. 4 is a diagram illustrating, for example, a floor surface on which a gait path is shown, in step S100 of a gait analysis method using an augmented reality interactive sports device according to an embodiment of the present disclosure.

FIG. 5 is a diagram illustrating, for example, a gait path on which a user moves, in step S100 of a gait analysis method using an augmented reality interactive sports device according to an embodiment of the present disclosure.

FIG. 6 is a diagram illustrating, for example, representation of a user position as the user moves, in a gait analysis method using an augmented reality interactive sports device according to an embodiment of the present disclosure.

FIG. 7 is a diagram illustrating the detailed flow of step S200, in a gait analysis method using an augmented reality interactive sports device according to an embodiment of the present disclosure.

FIG. 8 is a diagram illustrating, for example, a state indication image during a first measurement step, in a gait analysis method using an augmented reality interactive sports device according to an embodiment of the present disclosure.

FIG. 9 is a diagram illustrating measured positions of the user's feet, in a gait analysis method using an augmented reality interactive sports device according to an embodiment of the present disclosure.

FIG. 10 is a diagram illustrating the detailed flow of step S300, in a gait analysis method using an augmented reality interactive sports device according to an embodiment of the present disclosure.

FIG. 11 is a diagram illustrating, for example, a screen displaying a result of analysis, in step S400 of a gait analysis method using an augmented reality interactive sports device according to an embodiment of the present disclosure.

### DESCRIPTION OF THE REFERENCE NUMERALS IN THE DRAWINGS

100: augmented reality interactive sports device
110: body
120: projector part
130a, 130b: lidar sensor part
150: sensor connection part
170: communication part
180: display part
190: speaker part
200: gait analysis device
210: gait path generation part
220: gait measurement part
230: gait analysis part
240: index analysis part
S100: showing gait path on floor surface through augmented reality interactive sports device
S200: measuring foot position information of user moving along gait path by using lidar sensor part
S210: first measurement step of measuring foot position information of user moving along gait path by using lidar sensor part, and recording measured foot position information and measurement time
S220: second measurement step of measuring foot position information of user moving along gait path by using lidar sensor part, and recording measured foot position information and measurement time after first measurement step is terminated
S300: analyzing gait straightness and gait rhythm from foot position information of user and measurement time
S310: analyzing step length, stride length, step time, stride time, gait velocity, and number of steps per minute
S320: analyzing and graphing gait straightness
S330: analyzing and graphing gait rhythm

S400: analyzing gait stability index from gait straightness and gait rhythm and providing gait stability index

### Best Mode

Hereinafter, embodiments of the present disclosure will be described in detail with reference to the accompanying drawings such that the present disclosure can be easily embodied by those skilled in the art to which the present disclosure belongs.

However, the present disclosure may be embodied in various different forms and should not be limited to the embodiments set forth herein. Further, in order to clearly explain the present disclosure, portions that are not related to the present disclosure are omitted in the drawings, and like reference numerals designate like elements throughout the specification.

Throughout the specification, when a part is referred to as being “connected” to another part, it includes not only being “directly connected”, but also being “indirectly connected” by interposing the other part therebetween. In addition, when a part “includes” an element, it is noted that it further includes other elements, but does not exclude other elements, unless specifically stated otherwise, and is not to be understood as precluding the possibility that one or more other features, numbers, steps, operations, elements, parts, or combinations thereof may exist or may be added.

The embodiments described hereinafter are detailed descriptions of the present disclosure to facilitate understanding of the present disclosure and are not intended to limit the scope of the present disclosure. Thus, subject matter having the same scope and performing the same function as that of the present disclosure also falls within the scope of the present disclosure.

In addition, the elements, processes, steps, or methods included in the embodiments of the present disclosure may be shared as long as they do not technically conflict with each other.

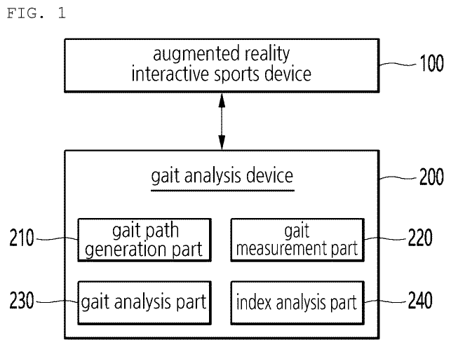

FIG. 1 is a diagram illustrating the configuration of a gait analysis device 200 using an augmented reality interactive sports device 100 according to an embodiment of the present disclosure. As shown in FIG. 1, a gait analysis device 200 using an augmented reality interactive sports device 100 according to an embodiment of the present disclosure may transmit and receive data to and from the augmented reality interactive sports device 100 configured to project a video onto a floor surface and track user motion by using a lidar sensor part to control interaction between content and a user. The gait analysis device may include: a gait path generation part 210 configured to show a gait path on the floor surface through the augmented reality interactive sports device 100; a gait measurement part 220 configured to measure foot position information of the user moving along the gait path by using the lidar sensor part configured to laser scan a plane at predetermined height from the floor surface; and a gait analysis part 230 configured to analyze gait straightness and gait rhythm from the foot position information of the user measured by the gait measurement part 220 and measurement time. The gait analysis device 200 may further include an index analysis part 240 configured to analyze a gait stability index from gait straightness and gait rhythm, and provide the gait stability index through the augmented reality interactive sports device 100. The gait analysis device 200 using the augmented reality interactive sports device 100 according to an embodiment of the present disclosure may be embedded in the augmented reality interactive sports device 100, or may communicate with the augmented reality interactive sports device 100 in a wired or wireless manner to analyze a user's gait and provide a result of analysis.

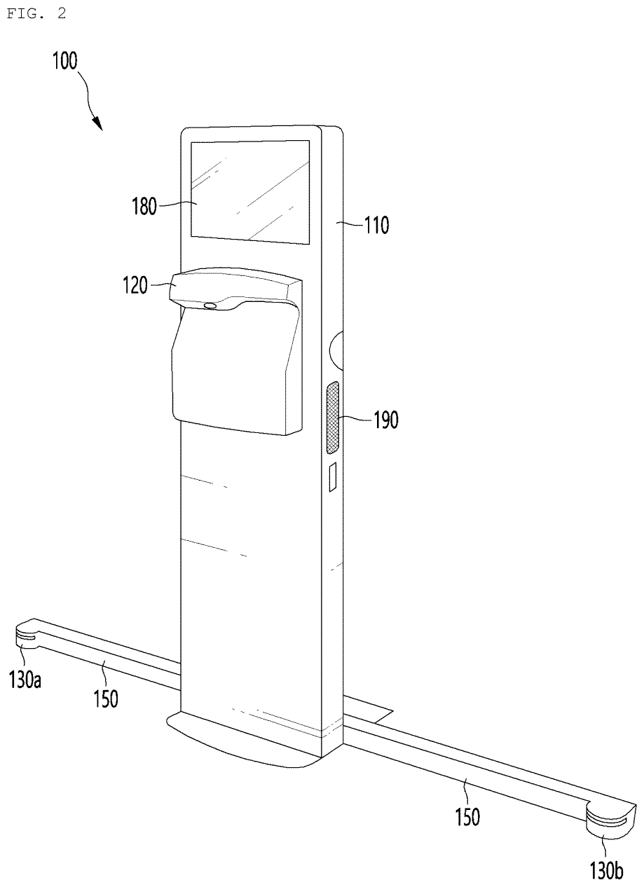

FIG. 2 is a diagram illustrating an augmented reality interactive sports device 100 used for a gait analysis method and device using an augmented reality interactive sports device 100 according to an embodiment of the present disclosure. As shown in FIG. 2, the augmented reality interactive sports device 100 may include: a body 110; a projector part 120 provided at a front surface of the body 110, and configured to project a video for sports content onto a floor surface; a lidar sensor part 130a, 130b installed facing a projection direction of the projector part 120; a motion tracking part (not shown) configured to track user motion by recognizing a user position according to the sports content through the lidar sensor part 130a, 130b; a sensor connection part 150 of which one end is connected to a side of the bottom of the body 110 and of which the other end is connected to the lidar sensor part 130a, 130b such that the lidar sensor part 130a, 130b is connected to the body 110 spaced apart therefrom by a predetermined distance; and a controller (not shown) configured to output the sports content through the projector part 120 to provide the user with the sports content based on augmented reality, and configured to control interaction between the sports content and the user by using the user motion tracked by the motion tracking part 140. The augmented reality interactive sports device 100 may further include: a display part 180 provided at the front surface of the body 110 and configured to receive a signal for selecting the sports content and output play information related to the sports content; and a speaker part 190 configured to output sound related to the sports content.

Herein, the lidar sensor part 130a, 130b may include two or more lidar sensors respectively connected to opposite sides of the bottom of the body 110 through the respective sensor connection parts 150, and may avoid tracking failures in laser scanning shade areas due to relative positions of a plurality of users and identify the position of each of the users. In addition, the motion tracking part 140 may use the lidar sensor part 130a, 130b to laser scan the plane at the predetermined height from the ground and recognize the positions of the user's feet, and may track the user motion by tracking changes in the positions of the user's feet.

Details of the augmented reality interactive sports device 100 may be configured with reference to Korean Patent No. 10-2233395 (title of invention: AUGMENTED REALITY INTERACTIVE SPORTS APPARATUS (100) USING LIDAR SENSORS, registration date: 23 Mar. 2021), and Korean Patent No. 10-2430084 (title of invention: AUGMENTED REALITY INTERACTIVE SPORTS SYSTEM USING LIDAR SENSORS, registration date: 2 Aug. 2022).

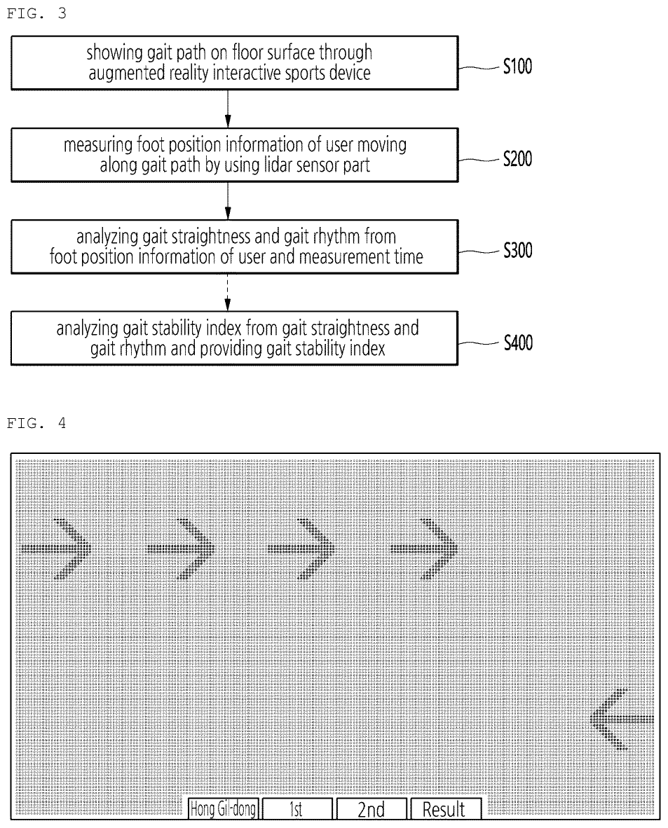

FIG. 3 is a diagram illustrating the flow of a gait analysis method using an augmented reality interactive sports device 100 according to an embodiment of the present disclosure. As shown in FIG. 3, a gait analysis method using an augmented reality interactive sports device 100 according to an embodiment of the present disclosure is embodied by the augmented reality interactive sports device 100 configured to project a video onto a floor surface and track user motion by using a lidar sensor part 130a, 130b to control interaction between content and a user. The gait analysis method may include: showing a gait path on the floor surface through the augmented reality interactive sports device 100 in step S100; measuring foot position information of the user moving along the gait path by using the lidar sensor part 130a, 130b in step S200; and analyzing gait straightness and gait rhythm from the foot position information of the user and measurement time in step S300. The gait analysis method may further include: analyzing a gait stability index from the gait straightness and the gait rhythm and providing the gait stability index in step S400.

In step S100, the gait path may be shown on the floor surface through the augmented reality interactive sports device 100. That is, the projector part 120 for projecting the content video onto the floor surface may be provided at the front surface of the augmented reality interactive sports device 100 as shown in FIG. 2. In step S100, the gait path may be shown by being projected onto the floor surface through the projector part 120. Herein, the gait path is a path designed for a user to follow for gait analysis, and may be intuitively shown as a guide line or an arrow.

FIG. 4 is a diagram illustrating, for example, a floor surface on which a gait path is shown, in step S100 of a gait analysis method using an augmented reality interactive sports device 100 according to an embodiment of the present disclosure. As shown in FIG. 4, in step S100 of the gait analysis method using the augmented reality interactive sports device 100 according to an embodiment of the present disclosure, the gait path may be represented by moving arrows on the floor surface. Representing the gait path by these moving arrows may prevent the user from intentionally walking while viewing a guide line in the case in which the path is represented by the guide line, and may encourage as much natural walking as possible. In addition, the moving arrows may enable the user to intuitively identify the gait direction.

The gait path shown in step S100 may be configured to pass, at least two times, the sensing area onto which the augmented reality interactive sports device 100 projects a video, the sensing area being laser scanned by the lidar sensor part 130a, 130b. More specifically, the gait path may be designed to start at one side of the sensing area and exit the sensing area and re-enter the sensing area. That is, the gait path is designed to pass the sensing area two or more times, thereby securing sufficient gait data.

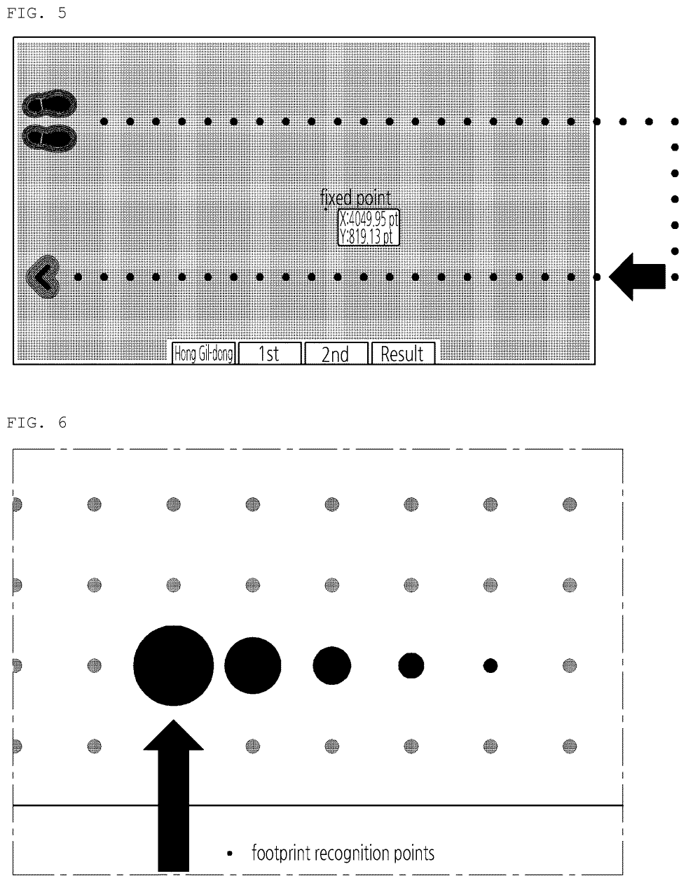

FIG. 5 is a diagram illustrating, for example, a gait path on which a user moves, in step S100 of a gait analysis method using an augmented reality interactive sports device 100 according to an embodiment of the present disclosure. As shown in FIG. 5, in step S100 of the gait analysis method using the augmented reality interactive sports device 100 according to an embodiment of the present disclosure, the gait path may be designed in a left-right reversed Korean letter ‘⊏’ shape that starts at an upper left portion of the sensing area and exits the sensing area and re-enters an lower right portion of the sensing area and ends at a lower left portion of the sensing area. Herein, the sensing area may be a space set for gait analysis within a space where the augmented reality interactive sports device 100 is able to accurately achieve video projection by the projector part 120 and user position measurement by the lidar sensor part 130a, 130b. In the example shown in FIG. 5, a space 4.5 m long and 2.5 m wide on the floor surface in front of the augmented reality interactive sports device 100 is set as the sensing area.

In step S200, the foot position information of the user moving along the gait path may be measured using the lidar sensor part 130a, 130b for laser scanning a plane at a predetermined height from the floor surface. More specifically, the lidar sensor part 130a, 130b of the augmented reality interactive sports device 100 may recognize the positions of the user's feet at a predetermined point from the ground, and the motion tracking part (not shown) may track the user motion by tracking changes in the positions of the user's feet. That is, the lidar sensor part 130a, 130b may laser scan a plane at a height of about 5 cm from the ground and detect the presence and positions of the user's feet, and may forward the detected foot position information to the gait analysis device 200. In particular, as shown in FIG. 2, since the lidar sensor part 130a, 130b includes two or more lidar sensors, two or more laser scanning areas overlap within the sensing area and the positions of the user's left foot and right foot may be accurately recognized.

Herein, in step S200, the measured foot positions may be shown by applying the measured foot position information in real time. More specifically, pixels may be set at preset intervals in the sensing area, and the measured foot position information may be used to show a preset recognition image at the pixel closest to the position of the user's foot.

FIG. 6 is a diagram illustrating, for example, representation of a user position as the user moves, in a gait analysis method using an augmented reality interactive sports device 100 according to an embodiment of the present disclosure. As shown in FIG. 6, in the gait analysis method using the augmented reality interactive sports device 100 according to an embodiment of the present disclosure, a sensing area may be divided into grid squares, and recognition images may be shown at pixels of grid points. Herein, the division of the sensing area into the grid squares may be configured such that the pixels pass through the gait path and the recognition image is equal to or smaller than the grid square in size. In addition, the recognition image in the shape of a white circle may be shown at the pixel closest to the foot position information of the user measured in step S200. Herein, a predetermined number (five in FIG. 6) of recognition images are shown starting from the current position of the user's foot in the direction of the path that the user has passed. The largest recognition image is shown at the current position of the user's foot. The further away from the current position, the smaller the size of the recognition image may be displayed.

These recognition images may give the user a sense that gait measurement is in progress and a sense of trust that his or her position is accurately measured. However, showing the positions of the user's feet too accurately may affect a natural gait, so the recognition images may be shown at preset pixels rather than the actual positions of the user's feet.

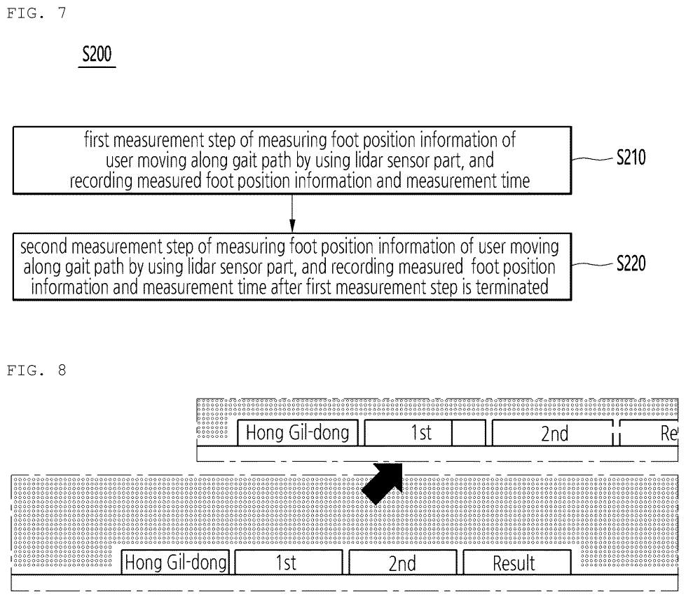

FIG. 7 is a diagram illustrating the detailed flow of step S200, in a gait analysis method using an augmented reality interactive sports device 100 according to an embodiment of the present disclosure. As shown in FIG. 7, step S200 of the gait analysis method using the augmented reality interactive sports device 100 according to an embodiment of the present disclosure may include: a first measurement step S210 of measuring the foot position information of the user moving along the gait path by using the lidar sensor part 130a, 130b, and recording the measured foot position information and the measurement time; and a second measurement step S220 of measuring the foot position information of the user moving along the gait path by using the lidar sensor part 130a, 130b, and recording the measured foot position information and the measurement time after the first measurement step is terminated.

The step S210 may be the first measurement step of measuring the foot position information of the user moving along the gait path by using the lidar sensor part 130a, 130b and recording the measured foot position information and the measurement time.

The step S220 may be the second measurement step of measuring the foot position information of the user moving along the gait path by using the lidar sensor part 130a, 130b and recording the measured foot position information and the measurement time after the first measurement step is terminated.

That is, in step S200, accurate gait data may be secured through the two measurement operations, the first measurement step and the second measurement step. People usually walk in various ways, such as dragging their feet, taking small steps so leaving their footprints overlapping, or walking with only part of their feet touching the ground, so it is necessary to extract valid information from the measured foot position information and analyze the valid information. In order to extract valid foot positions and analyze the same accurately, sufficient gait data needs to be secured. Therefore, measurement may be performed twice or more in step S200.

In the meantime, in step S200, a state indication image indicating a progress state of the first measurement step and the second measurement step may be shown.

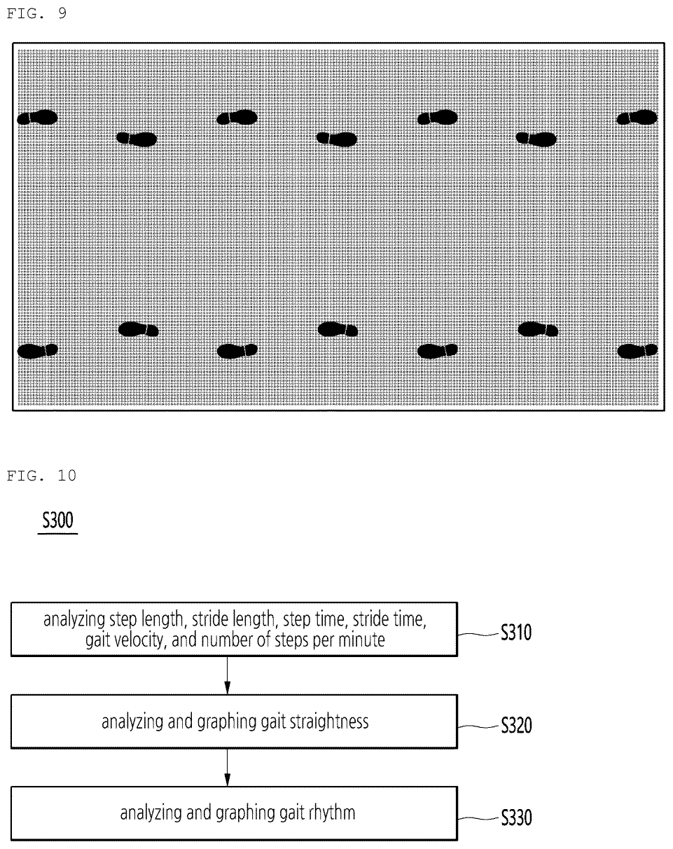

FIG. 8 is a diagram illustrating, for example, a state indication image during a first measurement step, in a gait analysis method using an augmented reality interactive sports device 100 according to an embodiment of the present disclosure. As shown in FIGS. 4 and 5, a menu for displaying information and operating the augmented reality interactive sports device 100 may be shown at one side of the video projected onto the floor surface. FIG. 8 is an enlarged view of the menu shown at one side of the sensing area, which may include an indication (Hong Gil-dong) of a person to be measured, indications (1st and 2nd) of the measurement steps, and an indication (result) of a result menu. Herein, the indications (1st and 2nd) of the measurement steps may function as state indication images indicating the progress states of the first measurement step and the second measurement step, respectively.

For example, to start the first measurement step, when the user steps on the menu button marked “1st” for a predetermined period of time or longer or kicks the menu button a predetermined number of times, the button is activated and the first measurement step may start. When measurement starts, the “1st” menu button flickers automatically as shown in the upper part of FIG. 8, or the color of the button gradually changes starting from left to right, thereby indicating the progress state of the first measurement step. When the first measurement step is terminated, the menu button marked “2nd” is automatically activated and the second measurement step may start. When the second measurement starts, the “2nd” menu button flickers or the color of the button gradually changes starting from left to right, thereby indicating the progress state of the second measurement step.

FIG. 9 is a diagram illustrating measured positions of the user's feet, in a gait analysis method using an augmented reality interactive sports device 100 according to an embodiment of the present disclosure. As shown in FIG. 9, in the gait analysis method using the augmented reality interactive sports device 100 according to an embodiment of the present disclosure, each time the first measurement step or the second measurement step is terminated, the measured positions of the user's feet may be shown as footprints. That is, during measurement, the positions of the user's feet are shown in an indirect way as shown in FIG. 6 because these may affect the user's gait. However, after measurement is completed, the foot positions are shown as soles-of-feet images in the sensing area on the floor surface so that the measured foot positions can be viewed at a glance as shown in FIG. 9, thereby increasing the user's trust.

In step S300, the gait straightness and the gait rhythm may be analyzed from the foot position information of the user measured in step S200 and the measurement time. FIG. 10 is a diagram illustrating the detailed flow of step S300, in a gait analysis method using an augmented reality interactive sports device 100 according to an embodiment of the present disclosure. As shown in FIG. 10, step S300 of the gait analysis method using the augmented reality interactive sports device 100 according to an embodiment of the present disclosure may include: analyzing a step length, a stride length, step time, stride time, a gait velocity, and the number of steps per minute in step S310; analyzing and graphing the gait straightness in step S320; and analyzing and graphing the gait rhythm in step S330.

In step S400, the gait stability index may be analyzed from the gait straightness and the gait rhythm analyzed in step S300, and the gait stability index may be provided through the augmented reality interactive sports device 100. Herein, in step S400, the gait stability index as well as a result of analysis, such as the gait straightness and the gait rhythm analyzed in step S300, may be provided. Herein, the result of analysis including the gait stability index may be provided through the projector part 120 or the display part 180 of the augmented reality interactive sports device 100.

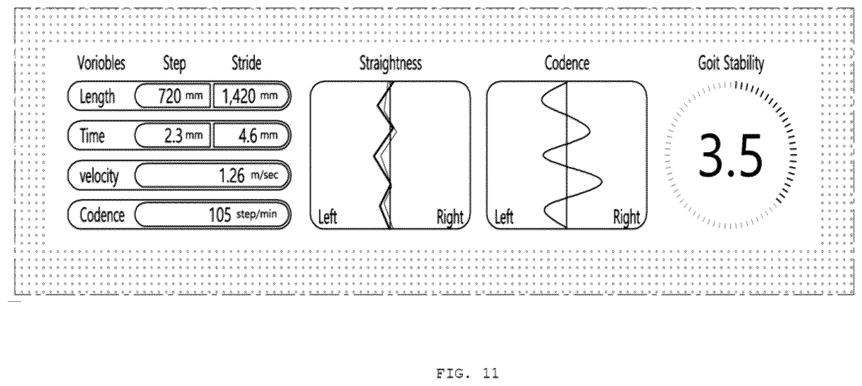

FIG. 11 is a diagram illustrating, for example, a screen displaying a result of analysis, in step S400 of a gait analysis method using an augmented reality interactive sports device 100 according to an embodiment of the present disclosure. As shown in FIG. 11, in step S400 of the gait analysis method using the augmented reality interactive sports device 100 according to an embodiment of the present disclosure, the step length, the stride length, the step time, the stride time, the gait velocity (the gait distance per second), and the gait rhythm (cadence, the number of steps per minute) may be provided. Herein, when the user steps on or kicks the “Result” button in the menu as shown in FIGS. 4, 5, and 8, a result of analysis as shown in FIG. 11 may be projected through the projector part 120 and provided. According to an embodiment, the display part 180 may display the step length, the stride length, the step time, the stride time, the gait velocity, and the gait rhythm in real time to provide measurement values varying as the user moves along the gait path.

Hereinafter, each step of step S300 of the gait analysis method using the augmented reality interactive sports device 100 according to an embodiment of the present disclosure will be described in detail with reference to FIGS. 10 and 11.

In step S310, the step length, the stride length, the step time, the stride time, the gait velocity, and the number of steps per minute may be analyzed from the foot position information of the user measured in step S200 and the measurement time. Herein, the step length may be the length taken with the left foot or the right foot in one step, and the stride length may be the length consisting of one step with the left foot and one step with the right foot. The gait velocity may be defined as the gait distance per second, and the number of steps per minute may represent gait rhythm.

In step S320, gait straightness may be analyzed by using the foot position information of the user measured in step S200 and may be graphed. A step or stride width may be defined as how far apart the left foot and the right foot are from the centreline of the gait, and this width is an important measurement value for a stable gait. In step S320, the foot position information of the user may be used to calculate the step or stride width, and the positions of the left foot and the right foot relative to the centreline of the gait may be graphed by being connected in time order of measurement. The generated gait straightness graph, such as the “Straightness” graph shown in FIG. 11, may be provided to the user, and data measured in the first measurement step and the second measurement step may be shown and provided in different colors.

In step S330, the gait rhythm may be analyzed on the basis of the step length and time analyzed in step S310 and may be graphed. The gait rhythm may be analyzed on the basis of the step length and the step time of the left foot and the right foot. More specifically, since the gait rhythm is defined as the number of steps per minute, the gait rhythm may be analyzed and graphed by calculating the number of steps per minute for the left foot and the right foot each. The generated gait rhythm graph, such as the “Cadence” graph shown in FIG. 11, may be provided to the user. The gait rhythm is information that may be used to determine the extent of use of both feet, and the gait rhythm may be understood by regarding the step length as horizontal movement during walking and the step time as vertical movement during walking. For example, when the left foot is uncomfortable, the length and the time taken with the left foot may be relatively short and the gait rhythm may lopsided.

In step S400, the gait stability index may be analyzed from the gait straightness and the gait rhythm, and may be provided to the user as “Gait Stability” shown in FIG. 11. The gait stability index may be provided as a score out of 5 calculated by combining the gait straightness and the gait rhythm.

In the meantime, a gait analysis method and device using an augmented reality interactive sports device 100 according to an embodiment of the present disclosure may store and manage measurement data, such as the measured foot position information of the user, and a result of analysis for each user. Therefore, the user may search or access his or her account to check his or her gait information history and establish an improvement direction or identify the degree of improvement.

As described above, according to a gait analysis method and device using an augmented reality interactive sports device 100 proposed in the present disclosure, a gait path is shown on a floor surface with the augmented reality interactive sports device 100 and foot position information of a user moving along the gait path is measured to analyze and provide gait straightness and gait rhythm, thereby performing gait analysis conveniently with ease and at low cost by using the augmented reality interactive sports device 100 used for various sports rather than an attached sensor or a controlled laboratory environment, and thus helping to identify poor gait habits and develop good gait habits.

In the meantime, an embodiment of the present disclosure may also be implemented in the form of a computer program stored in a computer-executable medium, recording or a medium including computer-executable instructions. A computer-readable medium may be any available medium that can be accessed by a computer, and may include all volatile and non-volatile media and removable and non-removable media. In addition, the computer-readable medium may include both a computer storage medium and a communication medium.

The computer storage medium may include all volatile and non-volatile, and removable and non-removable media implemented by any method or technology for storing information such as computer-readable instructions, data structures, program modules, or other data. The communication medium typically includes computer-readable instructions, data structures, program modules, other data in a modulated data signal, such as a carrier wave, or other transmission mechanism, and includes any information delivery medium.

Although the present disclosure has been described in connection with specific embodiments, some or all of the elements or operations of the embodiments can be realized using a computer system having a general purpose hardware architecture.

The foregoing description of the present disclosure is intended for exemplifications, and it will be understood by those skilled in the art that the present disclosure may be easily modified in other specific forms without changing the technical idea or essential features of the present disclosure. Therefore, it should be understood that the embodiments described above are illustrative in all aspects and not restrictive. For example, each element described as a single type may be implemented in a dispersed form, and likewise elements described as dispersed may be implemented in a combined form.

The scope of the present disclosure is characterized by the appended claims rather than the detailed description described above, and it should be construed that all alterations or modifications derived from the meaning and scope of the appended claims and the equivalents thereof fall within the scope of the present disclosure.

1. A gait analysis method using an augmented reality interactive sports device (100), the gait analysis method being embodied by the augmented reality interactive sports device (100) configured to project a video onto a floor surface and track user motion by using a lidar sensor part (130a, 130b) to control interaction between content and a user, the gait analysis method comprising:
(1) showing a gait path on the floor surface through the augmented reality interactive sports device (100);
(2) measuring foot position information of the user moving along the gait path by using the lidar sensor part (130a, 130b) configured to laser scan a plane at a predetermined height from the floor surface; and
(3) analyzing gait straightness and gait rhythm from the foot position information of the user measured in the step (2) and measurement time.

2. The gait analysis method of claim 1, wherein the gait path shown in the step (1) is configured to pass, at least two times, a sensing area onto which the augmented reality interactive sports device (100) projects the video, the sensing area being laser scanned by the lidar sensor part (130a, 130b).

3. The gait analysis method of claim 1, wherein in the step (2), gait measurement is performed multiple times, the step (2) comprising:
(2-1) a first measurement step of measuring the foot position information of the user moving along the gait path by using the lidar sensor part (130a, 130b) and recording the measured foot position information and the measurement time; and
(2-2) a second measurement step of measuring the foot position information of the user moving along the gait path by using the lidar sensor part (130a, 130b) and recording the measured foot position information and the measurement time after the first measurement step is terminated.

4. The gait analysis method of claim 3, wherein in the step (2),
a state indication image indicating a progress state of the first measurement step and the second measurement step is shown.

5. The gait analysis method of claim 1, wherein the step (3) comprises:
(3-1) analyzing a step length, a stride length, step time, stride time, a gait velocity, and the number of steps per minute from the foot position information of the user measured in the step (2) and the measurement time;
(3-2) analyzing the gait straightness by using the foot position information of the user measured in the step (2) and graphing the gait straightness; and
(3-3) analyzing the gait rhythm on the basis of the step length and the step time analyzed in the step (3-1), and graphing the gait rhythm.

6. The gait analysis method of claim 1, further comprising after the step (3),
(4) analyzing a gait stability index from the gait straightness and the gait rhythm analyzed in the step (3), and providing the gait stability index through the augmented reality interactive sports device (100).

7. A gait analysis device (200) using an augmented reality interactive sports device (100), the gait analysis device transmitting and receiving data to and from the augmented reality interactive sports device (100) configured to project a video onto a floor surface and track user motion by using a lidar sensor part (130a, 130b) to control interaction between content and a user, the gait analysis device comprising:
a gait path generation part (210) configured to show a gait path on the floor surface through the augmented reality interactive sports device (100);
a gait measurement part (220) configured to measure foot position information of the user moving along the gait path by using the lidar sensor part (130a, 130b) configured to laser scan a plane at a predetermined height from the floor surface; and
a gait analysis part (230) configured to analyze gait straightness and gait rhythm from the foot position information of the user measured by the gait measurement part (220) and measurement time.

8. The gait analysis device of claim 7, further comprising
an index analysis part (240) configured to analyze a gait stability index from the gait straightness and the gait rhythm, and provide the gait stability index through the augmented reality interactive sports device (100).
